(19) United States
(12) Patent Application Publication
(10) Pub. No.: US 2004/0033913 A1
(43) Pub. Date: Feb. 19, 2004

(54) Pearly luster concentrates

(76) Inventors: Gerd H Dahms, Duisburg (DE); Klaus Kwetkat, Bergkamen (DE)

Correspondence Address: C James Bushman, Browning Bushman, Suite 1800, 5718 Westheimer, Houston, TX, 77057-5771, US

(21) Appl. No.: 10/240,564

(22) PCT Filed: Apr. 2, 2001
(86) PCT No.: PCT/DE01/01246

(30) Foreign Application Priority Data
Mar. 31, 2000 (DE) 100 15 992.3

(51) Int. Cl. A61K 7/075

(52) U.S. Cl. 510/119; 510/120; 510/125

(57) ABSTRACT

This invention relates to aqueous, flowable pearly luster concentrates containing one or more pearlescent component(s), gemini surfactant(s), and at least one additional surfactant.

This invention relates to aqueous, flowable pearly luster concentrates containing one or more pearlescent component(s), gemini surfactant(s) and at least one additional surfactant.

Cosmetic shampoos and body cleaners, washing-up liquids, and liquid washing agents and cleansers frequently contain substances making these preparations pearlescent in order to improve the optical appearance of the wet surface and hence enhance the market values of the products. Many different substances are suitable to obtain this effect, e.g. fine-powdered natural materials, such as mica, pearl essence, inorganic materials, such as bismuth oxychloride and titanium dioxide pigments, metal salts of higher fatty acids, fatty acid glycol mono- or diesters, fatty acid alkanol amides or long-chain fatty alcohols.

Normally, flowable pearly luster concentrates which mostly contain organic pearlescent substances are employed for making commercially available consumer surfactant formulations. These pearly luster concentrates have the advantage of being incorporable cold into surfactant formulations, i.e. the whole preparation need not be heated to 60-80° C. to obtain the desired pearl lustre effect (cf. Crombie, R. L., Nicholson, S. H., Commun. Journal Com. Esp. Deterg., 28 (1998), p. 87-94). According to the prior art, these pearly luster concentrates can have the following composition:

1. A pearlescent organic component, e.g. fatty acid glycol ester or fatty acid glycerol ester of stearic acid,

2. dispersants, such as anionic surfactants (e.g. lauryl ether sulfate or fatty acid amidopropylbetaines),

3. crystallisation agents which are used according to the prior art to crystallise the pearlescent components in the desired form.

Irrespective of the components producing the pearlescent effect, it has been believed so far that the only effect and purpose of adding a pearl concentrate is to give the end product a pearlescent or silky gloss. It is desirable to produce this effect by using a pearl concentrate which is as easy to use as possible, i.e. a low-viscous, very stable pearly luster concentrate that does not adversely affect the properties of the end product. It is most important that the pearly luster concentrate does not contain any component irritating the skin or mucous membrane.

Further agents must be added to obtain additional properties required of many foaming and cleaning personal care preparations, e.g. lower irritation potential of the anionic principal surfactant in a preparation, improved initial foaming, washing liquor concentration, creaminess, and refatting. Active substances, such as vitamins, enzymes, and proteins, which can easily be reduced, oxidised or hydrolysed, are inappropriate for this use because they would require special pretreatment.

It would be possible to considerably simplify the formulations if many or all of the aforementioned functions were fulfilled by one of the components used in any case for making the surfactant composition. Furthermore, if active substances could be readily incorporated into any such preparation, this would open up technical flexibility which has so far been impracticable.

It has been surprisingly found that when using

surfactant compositions containing anionic gemini surfactants in conjunction with a poor-foaming anionic detergent component and/or electrolyte-stable anionic surfactants,

in combination with at least one nonionic surfactant and

a pearliser,

the pearly luster concentrate will become a multifunctional building component.

The present invention relates to aqueous, flowable pearly luster concentrates containing

(A) an anionic surfactant component consisting of

(A1) one or more anionic gemini surfactant(s) and one or more anionic detergent component(s) having a poor-foaming feature and/or

(A2) one or more electrolyte-stable anionic surfactant(s)

(B) one or more nonionic surfactant(s) and

(C) one or more pearl luster giving component(s) and, optionally, the following components:

(D) one or more active substance(s) and

(E) one or more stabiliser(s),

wherein each component is independently of one another present in the pearly luster concentrate in the following concentration, based on the total composition of the pearly luster concentrate:

5 to 30 wt % of component (A)

5 to 30 wt % of component (B) and

5 to 30 wt % of component (C)

10 to 60 wt % of water

0 to 50 wt %, preferably 0.1 to 10 wt %, most preferably 0.1 to 5 wt % of component (D) and

0 to 20 wt % of component (E).

The preferred embodiments of the invention are set out in the subordinate claims.

The anionic gemini surfactants (A1) are preferably used in conjunction with the poor-foaming anionic detergent component (A2) in the following proportions in the anionic surfactant component:

(A1) 10 to 80 wt %, preferably 20 to 60 wt %, or as much as 30 to 50 wt %, each referring to the sum of components (A1) and (A2), of one or more anionic gemini surfactant(s) and,

(A2) referring to the remainder, i.e. 90 to 20 wt % or 80 to 40 wt % or 70 to 50 wt %, each referring to the sum of components (A1) and (B1), of one or more poor-foaming, anionic detergent component(s).

The pearl concentrate is employed in quantities of 0.1 to 75 wt %, preferably 0.5 to 10 wt %, most preferably 1.0 to 5 wt % in the end product.

It has been surprisingly found that the pearlescent component in the formulation not only has the function of producing a pearlescent or silky gloss of high optical density and excellent brilliance, but that it can also reduce the irritation potential of other anionic surfactants, enhance initial foaming, produce foam containing much surfactant solution, and is very creamy. Moreover, the pearl concentrate of the invention can be formulated into products presenting good refatting properties and can be utilised as a vehicle for encapsulating and thus protecting the active components against oxidation, reduction, or hydrolysis, and for releasing them in a controlled way (e.g. odorants just at the right moment).

Formulations for cosmetic shampoos and toiletries, washing-up liquids, and liquid detergents can be remarkably facilitated by utilising the multifunctional pearliser of the invention because the pearliser is capable of taking on additional functions of other additives which will become unnecessary in this case or will be required in considerably reduced amounts, e.g. in the case of refatting components, foaming improvers, and irritation reducers. Cocoamidopropylbetaine and isethionates are given as examples.

Besides, when incorporating an active component, it will no longer be necessary to modify the whole formulation, as in the case of structured liquid detergents, because it is now possible to safely encapsulate the active component in the pearliser until it is released during the desired application.

When doing without a combination of gemini surfactant(s) and a component with a poor initial-foaming feature (according to German patent application 199 43 681.9) and using an electrolyte-stable, anionic surfactant instead (which is less preferred), the pearly luster concentrate formulated in this way has the added feature of encapsulated active components. However, it is preferable to employ the combined gemini surfactant/poor-foaming detergent component because this combination will better encapsulate the active component and will make the composition more stable.

Furthermore, the inventors found, without wanting to be tied to theory, that contrary to what has been described in literature (cf. Crombie, R. L.; Nicholson, S. H.; Commun. Journal Com. Esp. Deterg., 28 (1998), p. 87-94), the pearliser components in the pearlescent compositions of the invention are not present in the form of small crystals oriented in the surfactant double layers.

The pearl concentrate of the invention forms elongated, multilamellar, liquid-crystalline layers composed of several double layers of surfactant. When employed according to the invention, these elongated, multilamellar layers will remain unchanged in the application formulation or with the water concentrations typically used in such formulations, viz. 1 to 90 wt. %.

The elongated, multilamellar, liquid-crystalline layers formed in the compositions of the invention differ considerably from the vesicles made up of concentric membranes for encapsulating active components, which has been disclosed for example in WO 99/27907. The arrangement of the elongated, multilamellar, liquid-crystalline layers is unlike that of vesicles. It is rather like a system comprised of emulsifier and co-emulsifier forming a gel network (see Britto, J., EURO COSMETICS, 9 (1998), p.27-32).

FIG. 1/4 shows the form of the elongated, multilamellar, liquid-crystalline layers in the pearl concentrate referred to as formulation (A) in the examples. Said pearly luster concentrate was shock-frozen with liquid nitrogen and then broken. The fracture surface was coated with a conductive material and examined with a scanning electron microscope (ESEM) displaying the elongated, multilamellar structures which can be utilised for encapsulating active substances. These structures differ considerably from the spherical, onion-like structures of conventional compositions such as those described e.g. in WO 99/27907.

Active substances of at least moderately amphoteric character, i.e. having both hydrophobic groups and polar ones, which are incorporated as part of the double layers, are most suitable for encapsulating active substances in a pearlescent system. The active substances have no adverse effect on the pearliser efficiency. Oilsoluble active substances can be absorbed by the elongated, multilamellar, liquid-crystalline layers and are hence protected for example from oxidation, reduction, or hydrolysis. Water-soluble active substances can be incorporated in the water-containing interstitial layers between the elongated, multilamellar, liquid-crystalline ones. The protective effect of the pearly luster concentrate can be easily measured by determining the concentration of intact active substance during a certain period of time.

For the purpose of the present invention the term ‘gemini surfactants’ is defined as surface-active compounds consisting of at least (preferably) two surfactant units, i.e. one hydrophilic head group and one hydrophobic group interlinked with at least (preferably) one spacer in proximity to the head group. Gemini surfactants are also termed dimer surfactants because of their specific structure. There exist anionic, nonionic, cationic, and amphoteric gemini surfactants, depending on the kind of head group. However, in contrast to conventional surfactants, which are grouped in the same way, gemini surfactants can also bear combinations of different head groups, mostly of nonionic and ionic ones. Preferably, the gemini surfactant has at least one anionic group.

The preferred gemini surfactants used in the surfactant compositions of the invention have nitrogen atoms at the link between spacer, hydrophilic group, and hydrophobic group. More preferably, these gemini surfactants have spacers with amine or amide groups, but also spacers derived from dicarboxylic acids, betaine-derived hydrophilic double head groups, optionally presenting side groups obtained by alkoxylation, especially ethoxylation, which head groups may bear sulfonic acid, phosphonic acid, carboxylic acid, or alcohol groups, including polyalcohols, each of which having hydrophobic chains with 5 to 25 carbon atoms, which can be branched or unbranched and may bear up to two non-adjacent double bonds.

The following general variants of gemini surfactant structures as represented by the formulae hereinbelow are particularly useful for the surfactant compositions of the invention. The preferred structure variants are explained in detail by defining the substituents/spacers, wherein each definition of a substituent/spacer alone can characterise the gemini surfactant. Disclosure of the documents cited sub A.I through D.IV is hence expressly incorporated by reference herein with respect to the definition of the gemini surfactants.

Variant A: Structures Based on Amide- or Amine-Containing Spacers

A.I Gemini surfactants of the general formula (A.I) according to WO 96/14926

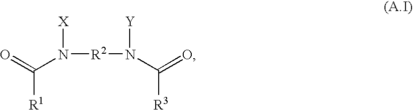

wherein the substituents have independently of one another the following meanings:

R¹, R³ C₅- to C₂₅-alkyl, branched or unbranched, saturated, optionally up to two times unsaturated if non-adjacent;

R² C₁- to C₁₂-alkylene;

X, Y (C₂H₄O—)ₓ(C₃H₆O—)_{y}—FR; x+y≧1, x: 0-15, y: 0-10; and

FR —SO₃M, —CH₂—CO₂M, —P(O) (OM)₂, H, —C₃H₆SO₃M; or —CH₂(CHOH)₄CH₂OH, insofar as x+y=0, wherein M=a counterion, such as alkali, (alkyl)ammonium, alkanol ammonium, H, or ½ alkaline earth.

A.II Gemini surfactants having dicarboxylic acid-based spacers of the general formula (A.II) in accordance with WO 96/25388

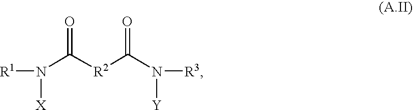

wherein the substituents have independently of one another the meanings as defined hereinabove by the general formula (A.I).

A.III Amphoteric gemini surfactants of the general formula (A.III) in accordance with WO 97/31890

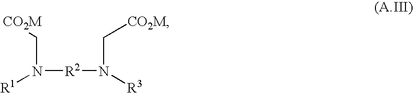

wherein the substituents have independently of one another the meanings as defined hereinabove by the general formula (A.I). Gemini surfactants of the general formula (A.III) are amphoteric compounds, which can turn into cationic ones if the ambient medium is acidic.

Variant B: Structures Based on Amide- or Amine-Containing Spacers

B.I Gemini surfactants of the general formula (B.I) in accordance with DE 19622612 or JP-A 10-175934

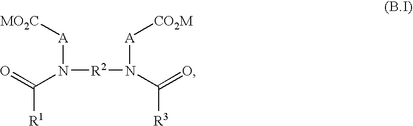

wherein the substituents have the following meanings:

R¹, R³ C₅- to C₂₅-alkyl, branched or unbranched, saturated, optionally up to two times unsaturated if non-adjacent;

R² C₁- to C₁₂-alkylene;

A CHR⁴, CH₂, C₂H₄, C₃H₆, C₄H₈;

R⁴ aminocarboxylic acid radical; and

M a counterion, such as alkali, (alkyl) ammonium, alkanol ammonium, H, or ½ alkaline earth.

B.II Gemini surfactants of the general formula (B.II) in accordance with EP 0 708 079

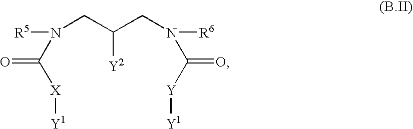

wherein the substituents have independently of one another the meanings as defined hereinabove by the general formula (B.I) and

R⁵, R⁶ represent C₆- to C₃₆-alkyl, branched or unbranched, saturated, optionally up to two times unsaturated if non-adjacent;

X is an alkylene- or-alkenylene group having 1 to 6 carbon atoms, which may be substituted with a hydroxyl group or a sulfonic acid group or a carboxy group;

Y¹ is a sulfonate- or sulfate group or a carboxyl group, and

Y² represents a hydroxyl group, a sulfuric acid residue, or —O—(CO)X—COOH.

B.III Gemini surfactants of the general formula (B.III) according to JP-A-8-311003

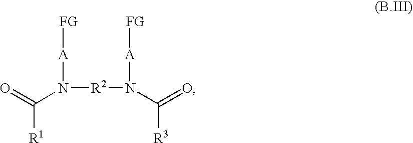

wherein the substituents have the meanings as defined hereinabove by the general formula (B.I) and

FG represents —COOM or —SO₃M.

B.IV Gemini surfactants of the general formula (B.IV) according to JP-A 11-60437

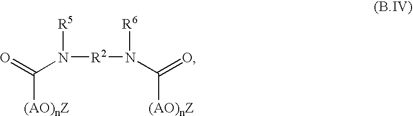

wherein the substituents have the meanings as defined hereinabove by the general formulas (B.I) and (B.II) and

AO represents alkylene oxide units, i.e. ethylene glycol-, propylene glycol-, and butylene glycol ether units, alone or arranged randomly or blockwise, wherein n=1 to 20, and

Z is —SO₃M, —C₂H₄SO₃M, —C₃H₆SO₃M, —P(O)(OM)₂ or —CH₂—COOM, —C₂H₄—COOM.

B.V Gemini surfactants of the general formula (B.V)

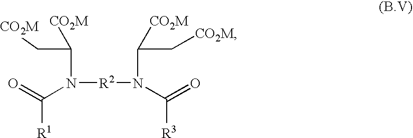

wherein the substituents have independently of one another the following meanings:

R¹, R³ C₅- to C₂₅-alkyl, branched or unbranched, saturated, optionally up to two times unsaturated if non-adjacent;

R² C₁- to C₁₂-alkylene;

M a counterion, such as alkali, (alkyl) ammonium, alkanol ammonium, H, or ½ alkaline earth, wherein the carboxylic acid groups may as well be neutralised only in part.

Variant C: Structures Based on Amide- or Amine-Containing Spacers

C.I Gemini surfactants of the general formula (C.I) according to EP 0 697 244,

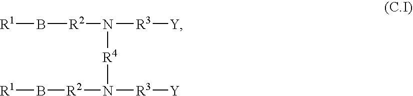

wherein the substituents have independently of one another the following meanings:

R¹ C₅- to C₂₅-alkyl, branched or unbranched, saturated, optionally up to two times unsaturated if non-adjacent, hydroxy-substituted or perfluorinated;

R² C₁- to C₁₂-alkylene or hydroxy-substituted derivatives thereof;

B an amide group [—C(O)N(R²)— or —N(R⁵)C(O)—], a carboxyl group [—C(O)O—or —OC(O)—], a polyether group [—(O(R⁶—O)ₓ—]

R⁵ C₁- to C₄-alkyl or hydroxy-substituted alkyl or H;

R⁶ C₂- to C₄-alkylene;

x a number from 1 to 20;

R³ C₁- to C₁₂-alkyl or hydroxy-substituted derivatives thereof, R⁷-D-R⁷ or a polyether group [—(O(R⁶—O)ₓ—];

R⁷ C₁- to C₆-alkylene or hydroxy-substituted derivatives thereof;

D —O—, —S—, —N(R⁸)—;

R⁴ alkylene or alkylaryl having from 1 to 12 carbon atoms or the hydroxy-substituted derivatives or R⁹-D¹-R⁹;

R⁸ C₁- to C₁₂-alkyl or hydroxy-substituted alkyl or H or R⁹-D¹-R⁹;

R⁹ C₁- to C₆-alkylene or hydroxy-substituted derivatives thereof or aryl;

D¹ —O—, —S—, —SO₂—, —C(O)—, [—(O(R⁷—O)ₓ—](R¹⁰)ₜ[N(R¹⁰)]_{z} or aryl;

R¹⁰ C₁- to C₁₂-alkyl or hydroxy-substituted alkyl or H or aryl;

t, z are independently of one another a number from 1 to 4, and

Y is independently of one another —SO₃H, O—SO₃H, —OP(O)(OH)₂, —P(O)(OH)₂, —COOH, —CO₂—C₆H₄—SO₃H and the salts thereof.

C.II Gemini surfactants of the general formula (C.II) according to EP 0 697 245

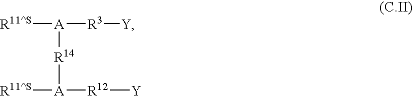

wherein the substituents have independently of one another the meanings as defined hereinabove by the general formula (C.I) and

R¹¹ is C₅- to C₂₃-alkyl, branched or unbranched, saturated, optionally up to two times unsaturated if non-adjacent, hydroxy-substituted or perfluorinated or R¹⁴—B—R²;

R¹⁴ is C₁- to C₁₂-alkyl, branched or unbranched, saturated, optionally up to two times unsaturated if non-adjacent, or the hydroxy-substituted derivatives;

R¹² means C₁- to C₁₂-alkylene, branched or unbranched, saturated, optionally up to two times unsaturated if non-adjacent, or the hydroxy-substituted derivatives, or an amide group [—C(O)N(R²)— or N(R⁵)C(O)—], a carboxyl group [—C(O)O— or —OC(O)—], a polyether group [—(O(R⁶—O)ₓ—] or R⁹—D¹—R⁹ and

A is —CR⁶═ or —N═, if whenever A is equal to —N═, R¹¹ represents R¹⁴—B—R².

C.III Gemini surfactants of the general formula (C.III) according to DE 4227391 and DE 19608117

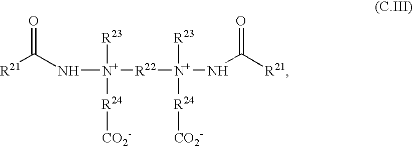

wherein the substituents have independently of one another the meanings as defined hereinabove by the general formulas (C.I) and (C.II) and

R²¹ represents C₅- to C₂₃-alkyl, branched or unbranched, saturated, optionally up to two times unsaturated if non-adjacent;

R²², R²⁴ are C₁- to C₆-alkylene;

R²³ is methyl, ethyl, propyl, or a polyether group [—(O(R⁶—O)ₓ—].

Variant D:

D. I Gemini surfactants of the general formula (D.I) according to U.S. Pat. No. 5,863,886

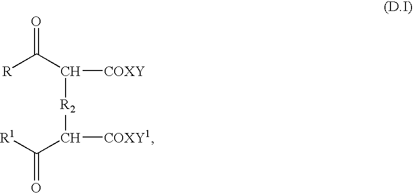

wherein the substituents have independently of one another the following meanings:

R, R¹ C₅- to C₃₀-alkyl, branched or unbranched, saturated, optionally up to two times unsaturated if non-adjacent, hydroxysubstituted or perfluorinated;

R² C₁- to C₁₀-alkylene, arylene, and hydroxy-substituted derivatives, a polyether [—O(R⁴O)ₓ—], —S—, —SO₂—, —O—, —S—S—, —O—R⁵—O—, or —S—R⁵—S—; variable for a direct bond between the two α-carbons;

R⁴ C₂- to C₄-alkylene;

R⁵ C₁- to C₁₀-alkylene, arylene or alkyl arylene, —N(R⁶)—, or —(NR⁶)—R⁷—(NR⁶)R⁶ C₁- to C₆-alkyl;

R⁷ C₁- to C₆-alkyl, wherein R⁷ and R⁶ may as well be part of a heterocyclic ring;

X polyether [—O(R⁴O)ₓ—] wherein x is a number from 1 to 30, —O—, NZ;

Z C₁- to C₁₀-alkyl, aryl, alkylaryl, or H, and

Y, Y¹ are independently of one another H, —CH₂—COOH and salts, a hydrocarbon radical having at least two hydroxyl groups, such as erythrose, threose, ribose, arabinose, xylose, fructose, lyxose, allose, altrose, glucose, mannose, galactose and mixtures thereof.

D.II Gemini surfactants of the general formula (D.II)

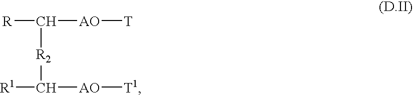

wherein the substituents have independently of one another the meanings as defined hereinabove by the general formula (D.I) and

AO means —C(O)—, —C(O)—(—O(R⁴O)ₓ—], —CH₂—[—O(R⁴O)ₓ—], —CH₂—O—;

T, T¹ are independently of one another —OM, —H, —CH₃, —C₂H₅, —SO₃M, —CH₂COOM, —C₂H₄—COOM, —C₃H₆—SO₃M, —O—P(O)(OM)₂ and

M is alkyli, ½ alkaline earth, ammonium, mono-, di-, trialkanolammonium, or H.

D.III Gemini surfactants of the general formula (D.III) according to WO 96/16930

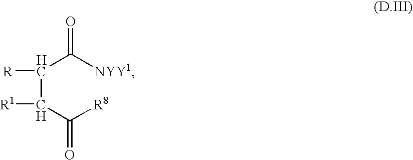

wherein the substituents have independently of one another the meanings as defined hereinabove by the general formulas (D.I) and (D.II) and

R⁸ is NYY¹, —O(R⁴O)ₓH or —O(R⁴O)ₓ—C(O)—CHR—CHR¹—C(O)NYY¹.

D.IV Gemini surfactants of the general formula (D.IV) according to WO 96/25384

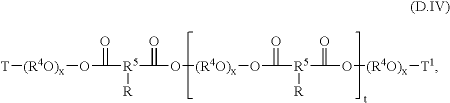

wherein the substituents have the meanings as defined hereinabove by the general formulas (D.I), (D.II), and (D.III) and

t is an integer from 1 to 100, preferably 1 to 20, most preferably 1 to 4.

The preferential detergent component characterised by poor foaming and preferably mildness to be employed in the surfactant compositions is chosen from among the following compounds:

water-soluble sugar surfactants, acylated protein derivatives, sulphosuccinates, especially sodium- -mono- and -dialkanol sulphosuccinates having branched or unbranched, saturated or mono- to triunsaturated if nonadjacent alkyl residues in the range of from C₆ to C₁8, or acyllactylates, especially sodium-, potassium-, magnesium-, or calcium salts of monomeric lactic acid esterified on the hydroxyl group with linear or branched, saturated or mono- to triunsaturated if non-adjacent, cyclic or acyclic C₆- to C₂₄-carboxylic acids, or its oligomers, the oligomerisation degree of the lactic acid being preferably from 1.1 to 10, most preferably from 1.1 to 4, or alkyl(poly)glucosides having an oligomerisation degree of from 1.0 to 10, preferably 1 to 3, and branched or unbranched, saturated or mono- to triunsaturated if non-adjacent, cyclic or acyclic alkyl residues having 6 to 24 carbon atoms, or alkali-, alkaline earth-, mono-, di-, and trialkanolammonium-, ammonium-, mono-, di-, trialkylammonium salts of N-acylated amino acids, optionally also including partially N-acylated oligo-/polyamino acids, e.g. alkyl isethionates which comprise alkyl residues with 6 to 24 carbon atoms and are branched or unbranched, saturated or mono- to triunsaturated if non-adjacent, or alkali-, alkaline earth-, mono-, di-, and trialkanolammonium-, ammonium-, mono-, di-, trialkylammonium salts of acylsarcosinates which comprise alkyl residues with 6 to 24 carbon atoms and are branched or unbranched, saturated or mono- to triunsaturated if non-adjacent, or protein condensates having C₆- to C₂₄acyl residues which are branched or unbranched, saturated or mono- to triunsaturated if non-adjacent, or betaines comprising alkyl chains with 6 to 24 carbon atoms, which can be branched or linear, saturated or mono- to tri-unsaturated if non-adjacent. Betaines of the amidoamine type are preferred. Acylglutamates with 6 to 24 carbon atoms in the acyl chain, which can be linear or branched, saturated or mono- to triunsaturated if non-adjacent, are also suitable. Further detergent components which are particularly preferred for use in the blends of the invention are acyllactylates and acylglutamates.

The term ‘mild’ employed herein means that the compounds/compositions are not subject to labelling, e.g. in accordance with EEC guideline 67-548, dangerous materials ordinance, with regard to the skin and eye irritation potential.

The term ‘poor-foaming feature’ employed herein means that the surfactants utilised as additional detergent components do not meet two out of the three criteria set forth hereinbelow for evaluating the foaming behaviour.

These criteria are:

lamellar thickness of the foam, in millimetres, immediately after foaming,

number of vesicles determined in a picture area enlarged a hundred times immediately after foaming,

both influenced by the microstructure of the foam, and

initial-foaming characteristics evaluated by a hand experiment.

Experiment for evaluating the poor-foaming feature of a product:

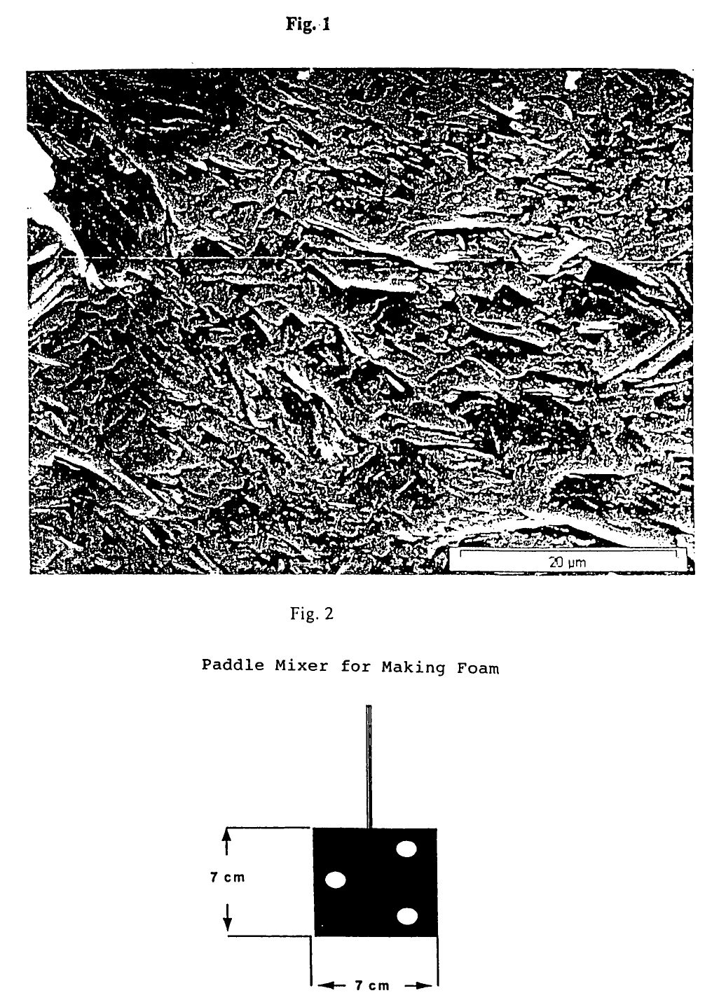

A portion (8 wt. %) of the surfactant under examination was dissolved in demineralised water. The surfactant solution was stirred for 10 minutes at 1,500 rpm using the paddle mixer depicted in FIGS. 2/4, 3/4, and 4/4. The solution was found to heat up slightly from room temperature to approx. 35° C. Stirring was terminated after 10 minutes. The foam produced in this way was skimmed off and promptly examined microscopically. The lamellar thickness was measured in millimetres, and the number of vesicles in the foam was determined in the image detail.

In addition to the foam quality produced by stirring, the initial foaming of a surfactant under running cold tap water was evaluated as well. In this test 2 grams of surfactant were spread over the palms and then evenly rubbed in under running water. The foam quality was evaluated by four grades: 0=no foaming, 1=moderate foaming, 2=good foaming, and 3=very good foaming.

Poor-foaming surfactants are those which do not fulfill at least two requirements of the three mentioned hereinbelow, namely

a lamellar thickness of less than or equal to 16 mm or

less than or equal to 16 vesicles in the image detail or

grade 1 or less in the foaming test.

Good-foaming surfactants fulfill all three requirements by the following values: lamellar thickness≧20 mm, vesicles in the image detail≧20 (both immediately after foaming), and grade 3 initial foaming.

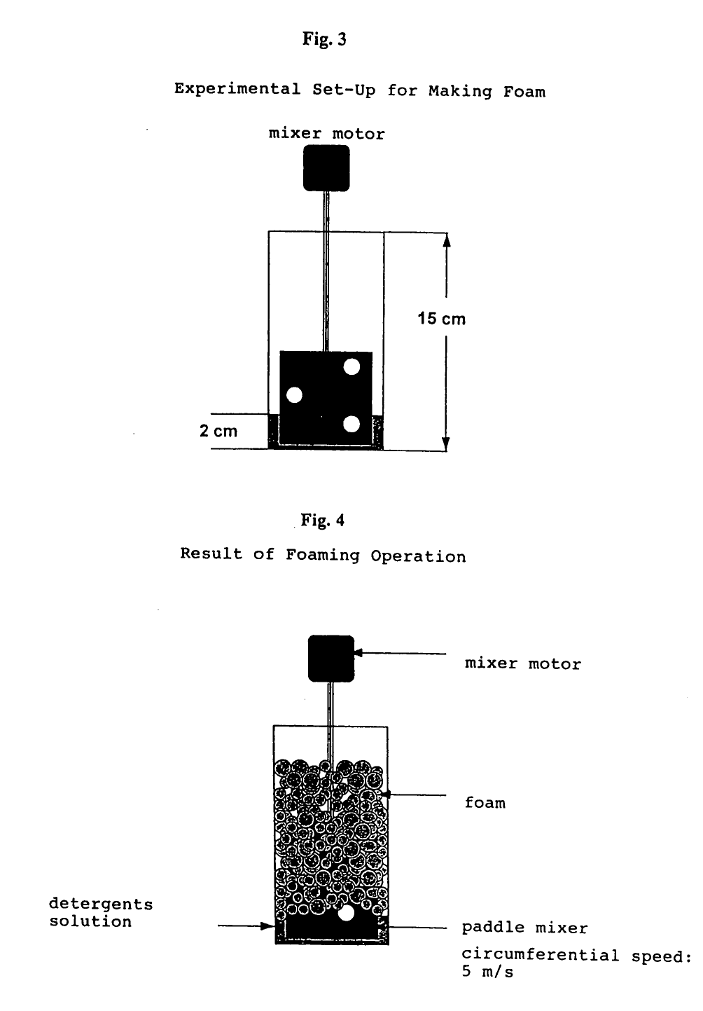

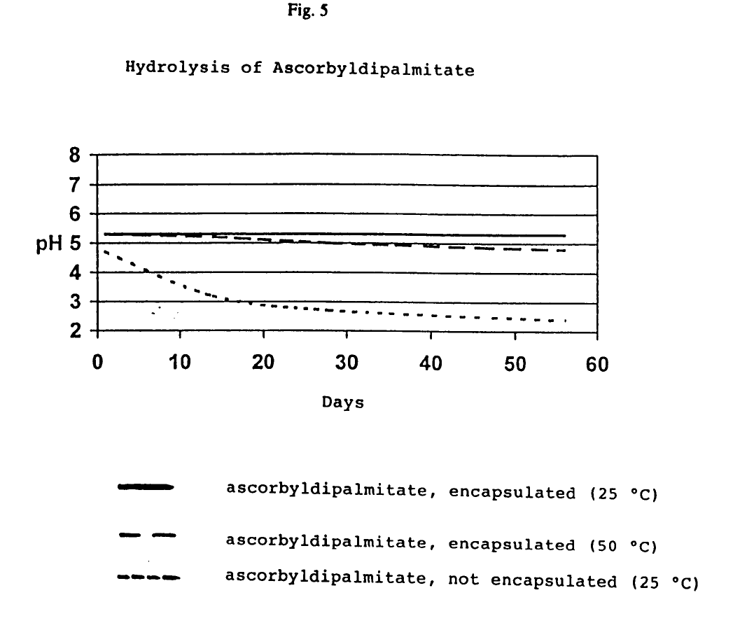

The method of making foam is illustrated in FIGS. 2/4 through 4/4. FIG. 2/4 shows the paddle mixer for making foam, FIG. 3/4 represents the experimental set-up for making foam indicating the heights in centimetres, wherein H is the height of the unfoamed solution, and FIG. 4/4 shows the result of the foaming operation at a circumferential speed of the paddle mixer of 5 m/s. After stirring for 10 minutes, the foam was skimmed off and microscopically examined after 2, 5, and 15 minutes. The experiments for evaluating the influence of the surfactants blends on the foaming behaviour were carried out using the experimental set-up described hereinbelow.

The method of making foam has been disclosed in German patent application 199 43 681 incorporated by reference herein.

Examples of electrolyte-stable anionic surfactants which are especially suitable for the pearl concentrate of the invention include alkyl sarcosinates, alkanol ether sulfates, alkanol sulfates, alkyl taurates and -isethionates, sulfobetaines, olefin sulfonates, ethercarboxylic acids and the salts thereof, and alkanolphosphoric acid esters and the salts thereof, wherein the alkyl chains have 8 to 22 carbon atoms.

The following nonionic surfactants are particularly suitable for making the pearly luster concentrates of the invention:

Addition products obtained by the addition of 2 to 30 moles of ethylene oxide and/or 0 to 5 moles of propylene oxide to linear alkyl alcohols having 6 to 22 carbon atoms, to fatty acids having 6 to 22 carbon atoms, to alkyl phenols having 8 to 15 carbon atoms in the alkyl group, and to mono-, di-, and triglycerides. C₁₂- to C₁₈fatty acid mono- and -diesters of addition products obtained by the addition of 1 to 30 moles of ethylene oxide to glycerol, sorbitan mono- and -diesters of saturated and unsaturated fatty acids having 6 to 22 carbon atoms and the ethylene oxide addition products thereof, alkyl mono- and -oligoglycosides having 6 to 22 carbon atoms in the alkyl residue and the ethoxylated analogues thereof. Addition products obtained by the addition of 2 to 60 moles of ethylene oxide to castor oil and/or dehydrated castor oil, polyol- and polyglycerol esters, such as polyglycerol polyricinoleate, polyglycerol dimerates, or polyglycerol poly-12-hydroxystearate, and mixtures of said classes of substances. Partial esters based on linear, branched, unsaturated or saturated C₆- to C₂₂fatty acids, ricinoleic acid, and 12-hydroxystearic acid, and glycerol, polyglycerol, pentaerythritol, dipentaerythritol, sugar alcohols (e.g. sorbitol), alkyl glucosides and polyglucosides (e.g. cellulose). Trialkyl phosphates and mono-, di-, and/or tri-PEG-alkylphosphates, alcohols of anhydrous lanolin, copolymers or the corresponding derivatives of polysiloxane-polyalkyl. Mixed esters of pentaerythritol, fatty acids, citric acid, and fatty alcohol and/or mixed esters of fatty acids having 6 to 22 carbon atoms, methylglucose and polyols, preferably glycerol and polyalkylene glycols.

Examples of particularly preferable products include alcohol ethoxylates having 6 to 13 carbon atoms and 4 to 15 moles of ethylene oxide, such as MARLIPAL® 013/120, Laureth-9, or MARLIPAL® 1012/6, and other nonionic surfactants with a similar HLB and a molecular mass of <800 g/mole.

The following compounds are particularly suitable as pearlising components in the pearly luster concentrate of the invention:

Metallic soaps of C₁₂- to C₂₂-fatty acids, e.g. magnesium stearate, Mg—, Zn—, Cu—, Al—, or Ti-salts in combination with one of the aforesaid fatty acids, C₁₂- to C₂₂-fatty acids, long-chain alkyl alcohols having 18 to 36 carbon atoms, alkyl alcohol ethers of C₈- to C₂₂-alkyl alcohols, fatty ketones with a total of at least 18 carbon atoms, alkanol amides based on C₁₀- to C₂₂-carboxylic acids and monoethanol amine, mono- or diesters of glycerol or ethylene glycol or 1,2-propylene glycol, butylene glycol, hexylene glycol, and/or polyethylene glycol having an average molecular weight of 80 to 1,000 grams/mole and C₁₂- to C₃₆-carboxylic acids. Mono- to triethylene glycol mono- and -distearates are preferred and the monostearates of mono-, di-, and triethylene glycol are most preferred.

The term ‘active substance’ according to the invention employed herein means substances that are especially sensitive to reduction or oxidation, especially difficult to dissolve in water and oil, or particularly sensitive to hydrolysis or surfactants. Examples of particularly suitable substances include antioxidants, vitamins (especially tocopherol, retinol) and the derivatives thereof, such as their esters, enzymes, proteins, and especially pheromones. Moreover, substances which may cause formulating problems due to their acidity, e.g. α-hydroxyacids, are appropriate as well. Also suitable for incorporation into the pearly luster concentrate are organic UV-absorbers, tanning agents, e.g. dihydroxyacetone, odorants, such as fragrances or insect repel lents, especially thiols, ternary or cyclic amines, natural essences the insect-repellent effects of which are known, e.g. citric or orange terpenes, neem oil extract, or insect-repellent pheromones, and essential oils, aromatisers or colorants.

When not using a gemini surfactant in combination with a poor-foaming detergent component, it may become necessary to add stabilisers to the lamellae if the active substances are insufficiently stable. Examples of especially suitable stabilisers include cholesterol and its derivatives, phytosterol and its derivatives, and saturated or unsaturated fatty acids, especially C₁₂- to C₂₂-fatty acids. Furthermore, the following hydrophilic compounds which will also improve the flow of the compositions of the invention can be employed as stabilisers in either case: glycerol or ethylene glycol or 1,2-propylene glycol, butylene glycol, hexylene glycol and/or polyethylene glycol having an average molecular weight of 80 to 1,000 g/mole.

Manufacture

The pearly luster concentrates of the invention can be manufactured by mixing the components (A), (B), (C), (D), and (E) at 20 to 80° C., preferably 40 to 50° C., and adding water at 30 to 80° C., preferably 40 to 50° C. The pearl lustre effect is produced at 25 to 30° C. and persists at temperatures of up to 60° C. The finished pearly luster concentrate can be admixed to the desired end formulation at room temperature.

The pearl lustre effect is rated from 1 (=brilliant) to 5 (=dull) and the state of crystallisation is examined microscopically (0=no crystals; 1=visible crystals). It is also examined whether the elongated, multilamellar layers are dissolved, wholly or in part, by the action of water or any of the surfactants (alcohol ether sulfates, cocoamidopropylbetaine) commonly used in end product formulations (1=yes, 0=no).

The temperature-dependent viscosity of the concentrates is determined to test the thermal stability of the concentrates.

The compositions in the following tables are examples of pearly luster concentrates of the invention.

| [CTFA INCI] Formulation | | |
|---|---|---|
| | A [wt. %] | B [wt. %] |
| PEG-3 stearate | 10 | 10 |
| Laureth-9 | 18 | 0 |
| Isotrideceth-13 | 0 | 18 |
| Sodium lauroyl lactylate, | 10 | 10 |
| sodium dicocoyl ethylene diamine | | |
| PEG-15 sulfate (gemini surfactant- | | |
| diamide ether sulfate) | | |
| (CERALUTION ® F) | | |
| PEG-400 | 8 | 8 |
| Aqua | 54 | 54 |

| | | |
|---|---|---|
| | C [wt. %] | [wt. %] |
| Glycol stearate | 10 | 10 |
| Laureth-9 | 18 | 0 |
| Isotrideceth-13 | 0 | 18 |
| Sodium lauroyl lactylate, | 10 | 10 |
| sodium dicocoyl ethylene diamine | | |
| PEG-15 sulfate (gemini surfactant | | |
| diamide ether sulfate) | | |
| PEG-400 | 8 | 8 |
| Aqua | 54 | 54 |
| [CTFA INCI] Formulation | E [wt. %] | F [wt. %] |
| PEG-3 stearate | 10 | 10 |
| Laureth-9 | 18 | 0 |
| Isotricleceth-13 | 0 | 18 |
| Sodium lauroyl lactylate, | 10 | 10 |
| sodium dicocoyl ethylene diamine | | |
| PEG-15 sulfate (gemini surfactant- | | |
| diamide ether sulfate) | | |
| PEG-400 | 8 | 8 |
| Aqua | 53 | 53 |
| Tocopherol | 1.0 | 0 |
| Retinol | 0 | 1.0 |
| [CTFA INCI] Formulation | G [wt. %] | |
| PEG-3 stearate | 10 | |
| Laureth- 9 | 20 | |
| Isotrideceth-13 | 0 | |
| Sodium lauroyl lactylate, | 10 | |
| sodium dicocoyl ethylene diamine | | |
| PEG-15 sulfate (gemini surfactant- | | |
| diamide ether sulfate) | | |
| PEG-400 | 8 | |
| Aqua | 49 | |
| Ascorbyl dipalmitate | 3 | |

The capability of the compositions of the invention to efficiently encapsulate active substances can be proved for example by the pH values measured on immersion of formulation G and the same amount of ascorbyl dipalmitate in water (see diagram 1/1). Hydrolysis of the ascorbyl dipalmitate is indicated by the significant decrease in pH value. When this ester is encapsulated, e.g. like in formulation G, no significant hydrolysis was observed.

[image]

1. An aqueous, flowable pearly luster concentrate containing
(A) an anionic surfactant component consisting of
(A1) one or more anionic gemini surfactant(s) and one or more anionic detergent component(s) having a poor-foaming characteristic and/or
(A2) one or more electrolyte-stable anionic surfactant (s)
(B) one or more nonionic surfactant(s) and
(C) one or more pearl luster giving component(s) and, optionally, one or both of the following components:
(D) one or more active substance(s) and
(E) one or more stabiliser(s).

2. The pearly luster concentrate of claim 1 containing (D) one or more active substance(s).

3. The pearly luster concentrate of claim 1 containing
5 to 30 wt % of component (A)
5 to 30 wt % of component (B)
5 to 30 wt % of component (C) and
10 to 60 wt % of water and, optionally,
0 to 50 wt %, preferably 0.1 to 10 wt % of component (D) and
0 to 20 wt % of component (E),
each based on the pearly luster concentrate.

4. A surfactant composition containing the pearly luster concentrate according to any one of the preceding claims in concentrations of from 0.1 to 75 wt %, preferably 0.5 to 10 wt %.

5. A pearly luster concentrate according to claims 1 to 3 or a surfactant composition according to claim 4,
characterized in that said concentrate has multilamellar, liquid-crystalline layers.
